(19) United States
(12) Patent Application Publication
(10) Pub. No.: US 2002/0038146 A1
(43) Pub. Date: Mar. 28, 2002

(54) Expandable stent with relief cuts for carrying medicines and other materials

(76) Inventor: Ulf Harry, Incline Village, NV (US)

Correspondence Address: Bruce H. Johnsonbaugh, Eckhoff, Hoppe, Slick, Mitchell & Anderson, 333 Market Street, Suite 3125, San Francisco, CA, 94105, US

(21) Appl. No.: 10/000,533

(22) Filed: Oct. 30, 2001

Related U.S. Application Data
(63) Continuation-in-part of application No. 09/774,760, filed on Jan. 30, 2001.
(63) Continuation-in-part of application No. 09/357,699, filed on Jul. 20, 1999.
(60) Provisional application No. 60/094,540, filed on Jul. 29, 1998.

(51) Int. Cl. A61F 2/06

(52) U.S. Cl. 623/1.16; 623/1.46

(57) ABSTRACT

An array of relief cuts is formed in at least some of the interconnected struts of an expandable stent. The relief cuts are sufficiently small to not significantly reduce the strength of each of the struts between flexion points, so that each strut between flexion points retains sufficient resistance against bending, twisting and buckling to perform its intended function. Relief cuts are provided which extend through the struts and either carry plugs of material such as medicine or other materials or, alternatively, one or both exterior surfaces of the stent may be covered with a medicinal or other coating. In one embodiment, a coating covers both the inner and outer surface of the stent and extends between the relief cuts, thereby connecting the layers of the coating and increasing its adhesion to the stent, itself. Multiple coatings may be applied to the stent. The relief cuts may be applied only at flexion points of the stent or only between flexion points of the stent or both. Coatings may be applied to some regions of the stent and not other regions of the stent. The preferred form of the invention uses relief cuts at flexion points to increase the width and reduce the thickness of individual struts and lattice support relief cuts are provided between the flexion points to increase the adhesion of a medicinal or other coating to the stent.

### CROSS REFERENCE TO RELATED APPLICATIONS

This is a continuation-in-part of U.S. patent application Ser. No. 09/774,760 filed Jan. 30, 2001 and entitled EXPANDABLE STENT WITH ARRAY OF RELIEF CUTS, and is also a continuation-in-part of U.S. patent application Ser. No. 09/357,699 filed Jul. 20, 1999 and entitled EXPANDABLE STENT, and claims the benefit of U.S. provisional application Serial No. 60/094,540 filed Jul. 29, 1998, entitled EXPANDABLE STENT.

### BACKGROUND AND BRIEF SUMMARY OF THE INVENTION

The present invention relates generally to balloon expandable and self-expanding stents capable of carrying medicines (and other materials) for use in blood vessels, the urethra and other body lumens. More particularly, the present invention provides one or more relief cuts formed in a stent to either carry a “plug” of medicine (for example) within each relief cut or to increase the adhesion of a medicinal coating (for example) applied to the surface of the stent. The present invention allows stents to carry various materials, including medicines, lubricants, chemicals and radioactive materials. According to one form of the present invention, the relief cuts are strategically placed to provide a “support lattice” affording increased adhesion of medicinal coatings. The present invention in its preferred form allows the use of wider and thinner struts, while simultaneously providing the presence of relief cuts for lattice-support to increase adhesion of medicinal coatings.

The present invention also facilitates the use of multiple layers of different types of medicines or combinations of different materials in a multi-layered coating. Alternatively, different regions of the stent surface may be coated with different materials.

The preferred form of the invention provides a coated stent having a reduced overall wall thickness compared with prior art non-coated stents. This is achieved by the use of “flexion” relief cuts which in turn allows the use of much wider and thinner struts. The wider and thinner struts provided by this invention are very resistant to twisting or warping, since each strut retains its width at its flexion points. In contrast, some prior art stent designs increase flexibility of the stent by significantly reducing the strut width at flexion points; significant disadvantages of the prior art approach are increased cost and increased tendency of the struts to twist or warp as the stent expands.

The relief cuts of the present invention are sufficiently small so that the structural strength of each strut between flexion points is not significantly reduced, as compared with the same strut without relief cuts. The word “strut” is used broadly herein, and is used to refer to one of a series of interconnected members wherein those interconnected members flex at flexion points as the stent expands. The present invention provides relief cuts at either the “flexion” points and/or in the strut between flexion points. Although the present invention uses relief cuts at flexion points to allow stents to expand with less pressure, and extra relief cuts may be formed between flexion points, the strength of each strut or interconnected member between flexion points is not significantly reduced. That is, each strut (or interconnected member) does not significantly lose its resistance to bending, twisting or buckling between flexion points because of the presence of relief cuts according to the invention.

A significant aspect of the present invention is that selective placement of an array of “flexion” relief cuts at strategic locations on a stent allows the stent to expand with less pressure in a predetermined and controlled non-uniform fashion while simultaneously allowing selective placement of “support lattice” relief cuts to increase adhesion of medicinal coatings without significantly reducing strut strength between flexion points. For example, “flexion” relief cuts in one embodiment are utilized only at the distal and proximal end regions of a dogbone-shaped stent, which causes the end regions to expand first, with the central region of the stent expanding last; while simultaneously, “support lattice” relief cuts are provided in the central region of the stent to maximize the adhesion of medicinal coatings to the central region. As a further example, combinations of “flexion” and “support lattice” relief cuts may be applied in various patterns to cause stents to act differently; some patterns allowing stents to be used better in curved and tapered vessels or lumens, and some patterns allowing the stent to bend more easily in a given direction.

Another advantage of the present invention is that the “flexion” and/or “support lattice” relief cuts may be applied together with coatings to a variety of existing and commercially successful balloon expandable and self-expanding stent designs. The use of the “flexion” and/or “support lattice” relief cuts as described and claimed herein can quickly provide existing commercial stents with most of the advantages of the present invention.

Another aspect of the present invention is that relief cuts may be utilized which perform a dual function; a single relief cut can add increased flexibility to the stent while simultaneously increasing the adhesion of a medicinal (or other) coating. For example, according to the invention, a prior art stent may be modified by having relief cuts formed only at its flexion points; when the modified stent is thereafter coated with a medicinal coating (for example), the relief cuts increase the adhesion of the coating to the stent.

It is therefore a primary object of the present invention to provide one or more relief cuts in an expandable stent to increase the adhesion of a medicinal or other coating applied to the stent.

Another object is to provide a stent having relief cuts and being coated with multiple layers of different materials, or to apply different coatings to several regions of a single stent.

Another object of the invention is to provide an array of “flexion” relief cuts in prior art as well as new stent designs to allow those stents to expand more easily and with less pressure than is the case in the absence of relief cuts and to simultaneously provide increased adhesion of medicinal (or other) coatings to the stent.

Still another object of the invention is to provide a balloon expandable and self-expandable stent design having an array of “flexion” relief cuts which, not only increase adhesion of coatings, but also allow the use of wider and thinner members in the stent to increase the radio-opacity and vessel wall coverage of the stent; a related object is to add “support lattice” relief cuts to further increase adhesion of a medicinal coating or other coating to the stent surface.

Still a further object of the invention is to provide a medicinally coated stent having “support lattice” relief cuts together with an array of “flexion” relief cuts which not only allows the use of wider members, but also allows the use of thinner wall stents, thereby increasing the effective inner diameter of arteries and other lumens carrying those stents. The use of thinner walled stents minimizes the profile or cross section of the stent and provides more clearance in inserting and deploying the stent.

A still further object of the invention is to provide one or more relief cuts to a stent, wherein each relief cut carries a “plug” of medicine or other material.

Another object is to provide a coated stent with relief cuts, wherein the surface coating dissolves into the vessel wall and thereafter the “plugs” of material carried within the relief cuts dissolve into the vessel wall.

Other objects and advantages of the present invention will become apparent from the following description and the drawings wherein:

### BRIEF DESCRIPTION OF THE DRAWINGS

FIG. 1 is a perspective view of a prior art stent cell configuration shown in its expanded state;

FIG. 2 is a perspective view showing the prior art stent cell design of FIG. 1 as modified by the present invention, showing a much wider and thinner strut and showing a plurality of relief cuts, but before any coating has been applied to the stent;

FIG. 3 is a perspective view of the stent cell illustrated in FIG. 2 after a medicinal coating has been applied to the surface of the stent and into each of the relief cuts;

FIG. 4 is a section on the line 4-4 of FIG. 3;

FIG. 5 is a section on the line 5-5 of FIG. 3;

FIG. 6A is a sectional view of a portion of the stent similar to that shown in FIG. 4 but wherein a second separate layer of coating has been applied to the stent

FIG. 6B is a sectional view of a portion of the stent similar to that shown in FIG. 4 but wherein an alternate second separate layer of coating has been applied to the stent;

FIG. 7A is a schematic illustration of the prior art stent cell configuration shown in FIG. 1 illustrating the use of “lattice support” relief cuts along with a medicinal coating but wherein no “flexion” relief cuts have been applied;

FIG. 7B is a schematic illustration of the cell shown in FIG. 1 wherein “flexion” cuts and a medicinal coating are added, but no relief cuts between flexion points;

FIG. 8 is a schematic illustration of a dogbone stent in its unexpended position wherein the horizontal dash lines at the distal and proximal ends represent the placement of flexion relief cuts and wherein the small “O's” in the central region illustrate a plurality of “lattice support” cuts and wherein the central region is coated with a medicine;

FIG. 9 is a schematic illustration of the dogbone stent of FIG. 7 shown in its expanded position in an artery wherein the central section of the stent has been expanded into contact with a plaque deposit and wherein the medicinal coating contacts the plaque deposit;

FIG. 10 is a plan view of an alternate stent cell design wherein a plurality of flexion relief cuts are shown carrying plugs of medicine or other materials;

FIG. 11 illustrates an alternate form of the invention wherein the general cell configuration shown in FIG. 9 is illustrated but wherein “lattice support” relief cuts are applied to the stent in addition to an array of flexion relief cuts;

FIG. 12 is a schematic illustration of an alternate form of the invention as applied to yet another stent cell configuration;

FIG. 13 is a sectional view of an alternate relief cut design having a tapered shape and filled with a “plug” of medicine;

FIG. 14 is a schematic illustration of an inclined relief cut illustrating a coating of medicine applied to the exterior surfaces of the stent and filling the inclined relief cut;

FIG. 15 illustrates how the present invention may be applied to the cell configuration of FIG. 1 with a series of relief cuts, some of which increase flexion of the stent and simultaneously provide lattice support for the coating;

FIG. 16 illustrates another embodiment of the invention wherein the “lattice support” relief cuts are considerably smaller in dimension than the “flexion” relief cuts;

FIG. 17 illustrates a further embodiment of the invention wherein the “lattice support” relief cuts are applied closer to the edges of each strut and off the centerline of the strut; and

FIG. 18 is a schematic illustration of a tapered stent wherein the central portion only of the stent is coated with a medicinal coating pursuant to the present invention.

### DETAILED DESCRIPTION OF THE DRAWINGS

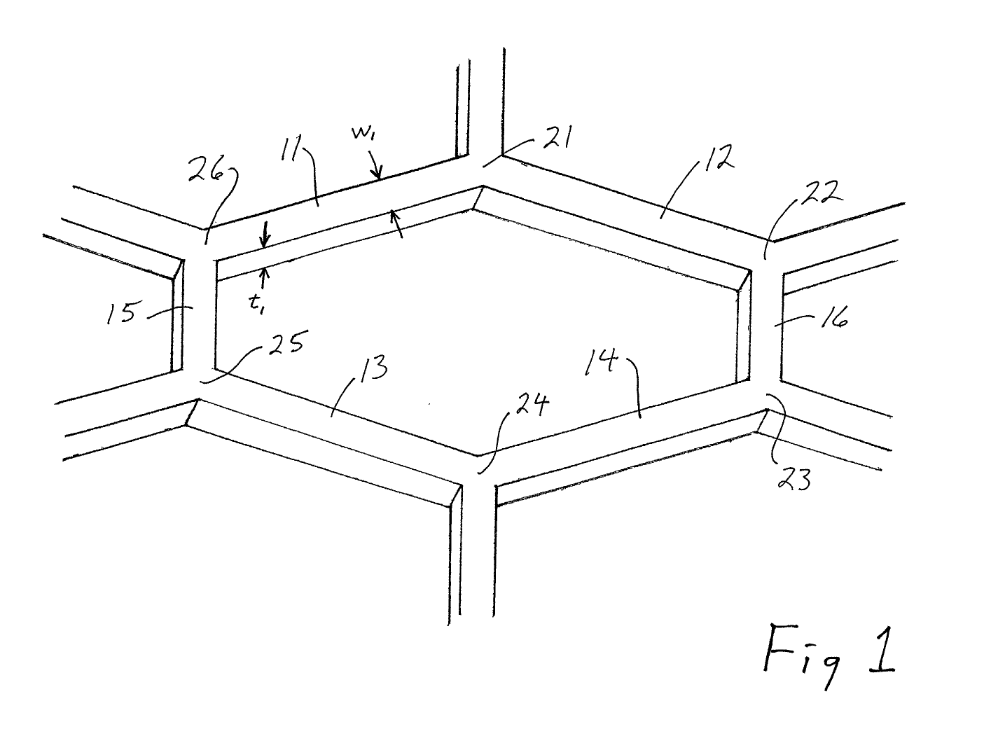

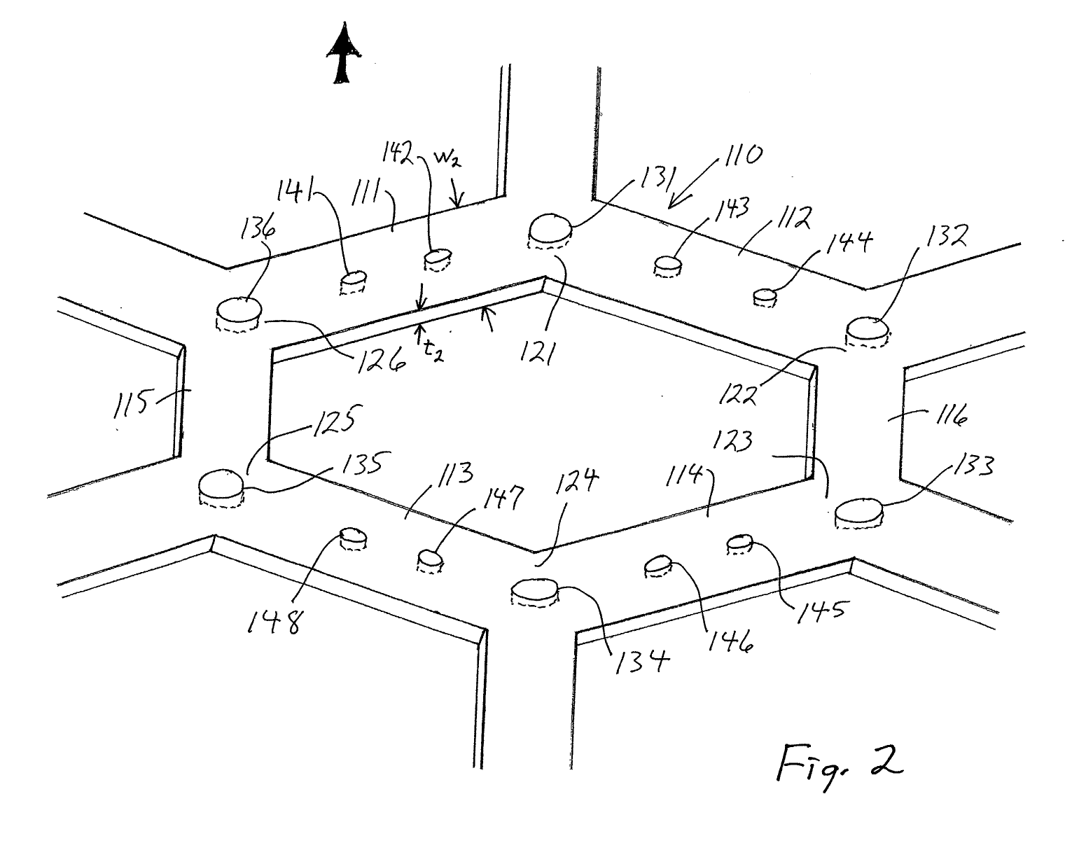

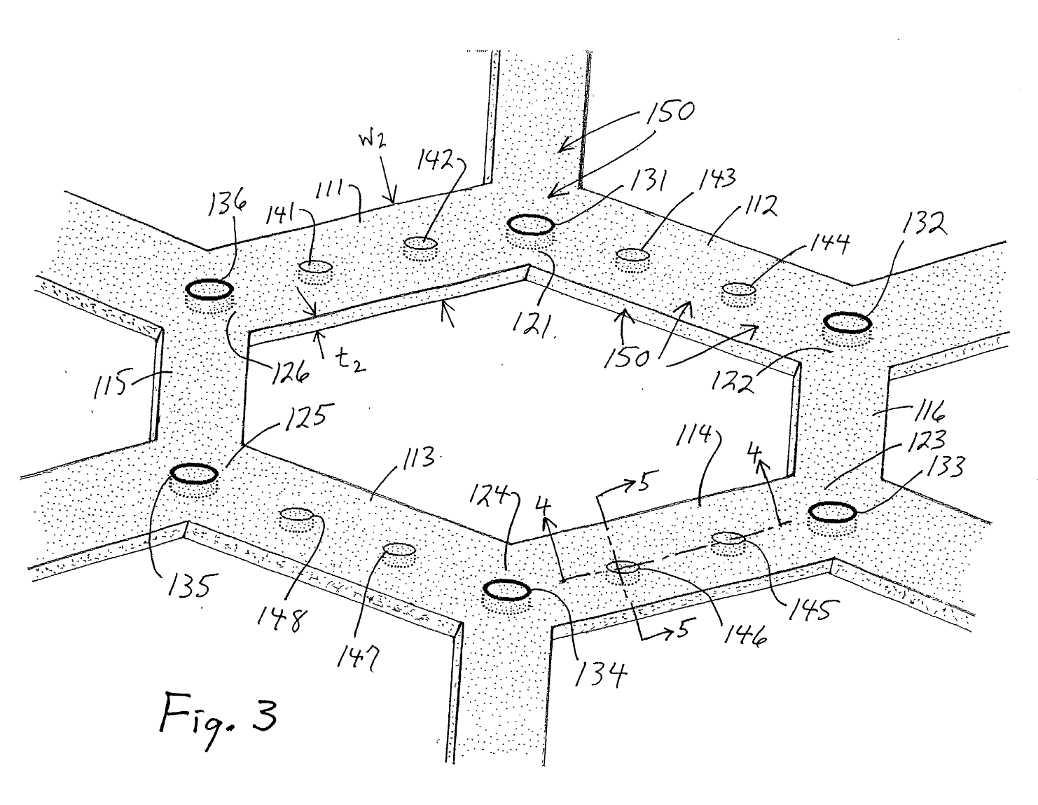

The preferred form of the present invention is best illustrated by comparing the prior art stent cell configuration of FIG. 1 with that same cell configuration as modified by the present invention and illustrated in FIGS. 2 and 3. FIG. 1 illustrates an expanded cell of the Palmaz U.S. Pat. No. 4,739,762. The cell shown generally as 10 includes four struts 11, 12, 13 and 14 that, in their unexpended position, extend parallel to the longitudinal axis of the stent. Struts 15 and 16 extend in a direction perpendicular to the longitudinal axis of the stent. Stent cell 10 has a series of six “flexion” points 21-26, each of which is located at a juncture of two adjacent struts. As the stent cell 10 is expanded by a balloon, the individual struts 11-16 remain essentially straight, and the cell expands by flexing at each of “flexion” points 21-26. The prior art stent cell configuration 10 utilizes struts that have cross sections that are essentially square and having a width w₁ and thickness t₁ that are approximately equal.

FIG. 2 illustrates the present invention as applied to the stent cell configuration of FIG. 1. As shown in FIG. 2, stent cell shown generally as 110, has six struts 111-116 that extend in the same directions as corresponding struts 1 1-16 in the unexpended position of cell 110. The present invention includes a series of “flexion” cuts 131-136 that are formed in the stent in the ends of struts 111 -116 at the series of flexion points 121-126, each of which is located at a juncture of adjacent struts of cell 110. Each of the “flexion” cuts 131-136, as shown in FIG. 2, is cylindrical in design and extends through the entire thickness t₂ of the stent material. The purpose of the “flexion” cuts is to allow the stent cell 110 to expand in response to significantly less pressure which in turn allows the use of a significantly wider strut dimension w₂ and a significantly reduced strut thickness t₂, which feature is explained in full detail in U.S. patent application Ser. No. 09/774,760, referred to above and incorporated herein by reference as though set forth in full. In addition to the array of “flexion” relief cuts, an array of “lattice support” relief cuts 141-148 are provided in the four struts 111-114. The purpose of these “lattice support” relief cuts is to increase the adhesion of a coating material which may be applied to the stent, as for example by dipping the stent into the coating material or spraying the coating material onto the stent or otherwise. The “lattice support” relief cuts extend through the thickness t₂ of each of the struts 111-114 and are positioned away from the flexion points 121-126 so they do not significantly affect the pressure required to expand the stent and do not appreciably alter the shape of cell 110 as it expands. As shown in FIG. 2, each of the “flexion” relief cuts 131-136 and each of the “lattice support” relief cuts 141-148 are positioned equidistantly from the edges of the stent struts. The purpose of positioning the relief cuts generally along the center of the struts is to maintain the widest possible connection between struts at the points of flexion 121-126. Maintaining the widest points of connection maximizes the resistance of each of the struts 111 -116 to twisting or warping as the stent expands. This is a distinct difference over some prior art stent designs that increase the flexibility of the stent by grinding or otherwise forming the flexion points to be significantly narrower than the width of the struts. Those designs tend to experience twisting or warping of the individual struts as the cell is expanded.

The presence of relief cuts 131-136 and 141-148 in struts 111-116 does not significantly reduce the strength of the individual struts between flexion points, i.e., each strut retains enough of its resistance to bending, twisting and buckling between flexion points to properly function in its intended environment. Relief cuts 131-136 formed in the ends of struts 111-116 do increase the flexibility of struts at the flexion points 121-126 without significantly reducing strut strength between flexion points.

FIG. 3 illustrates the stent cell configuration of FIG. 2 after a medicinal coating 150 has been applied by dipping the stent. The medicinal coating 150 is applied to all surfaces of the stent and completely fills all the relief cuts. It is within the scope of the invention to coat the entire stent with materials other than medicines as noted above.

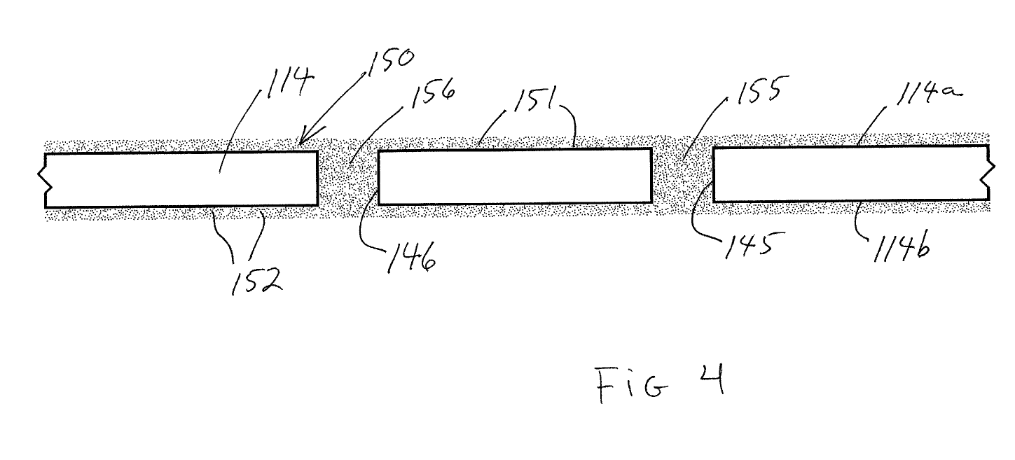

FIG. 4 is a side elevational view in section of strut 114 showing relief cuts 145 and 146 that extend through strut 114. The medicinal coating 150 is shown having an upper layer 151 covering the upper (or outer) surface 114a of strut 114 and a lower layer 152 that covers the lower (or inner) surface 114b of strut 114. The upper layer 151 and lower layer 152 are connected by “plugs” of material 155 and 156 that fill the relief cuts 145 and 146. The upper layer 151 forms the outer layer when the cylindrical stent expands, and lower layer 152 forms an inner layer when the stent expands. The plugs 155 and 156 connect the upper surface 151 and lower surface 152 and form a “support lattice” which greatly enhances the adhesion of the coating 150 to each individual strut, such as strut 114, and greatly increases the adhesion of the coating 150 to those portions of the entire stent that contain relief cuts in accordance with the present invention. The presence of relief cuts, particularly in the struts that are subjected to the most flexion and bending during expansion of the stent, i.e., struts 111-114, significantly reduces the likelihood of the coating separating from the surface of the struts as the stent is expanded. Furthermore, the “plugs” 155 and 156 provide additional material to dissolve into the vessel wall. If coating 150 is a dissolvable medicine, after the outer surface 151 dissolves, plugs 155 and 156 dissolve and extend the time period during which medicine is applied.

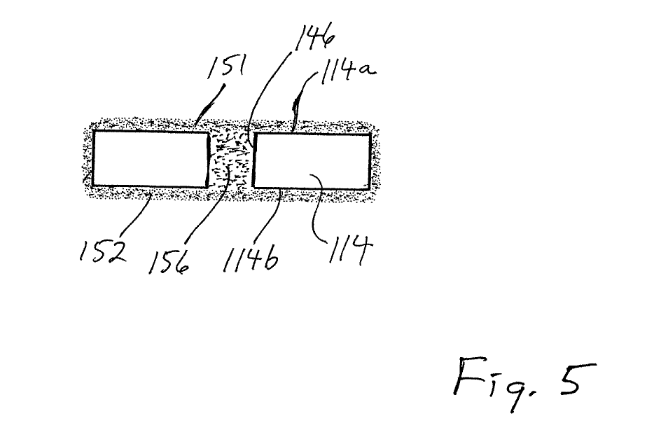

FIG. 5 is a sectional view on the line 5-5 of FIG. 3 showing relief cut 146 and illustrates how “plug” 156 of the medicinal coating 150 connects the upper (or outer) layer 151 with lower (or inner) layer 152.

It is also within the scope of the invention to apply the coating by spraying the outer surface of the stent, and allow the sprayed coating to extend into and through the relief cuts, without coating the inner surface of the stent.

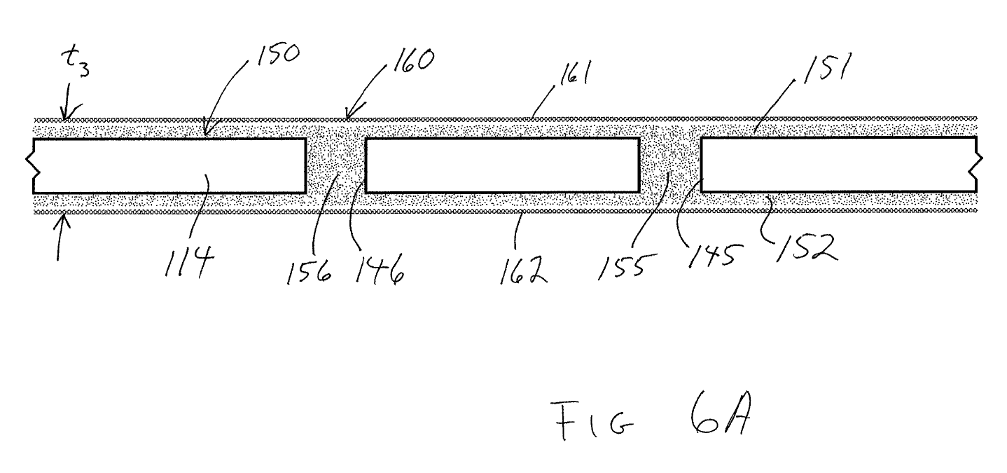

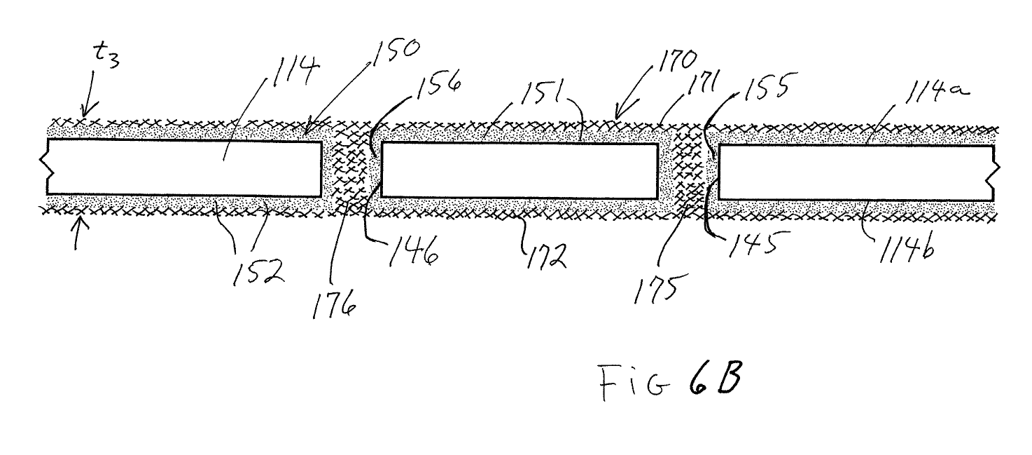

FIG. 6A illustrates an alternate embodiment of the invention wherein a second coating 160 is applied directly on top of first coating 150. Second coating 160 may also be applied by dipping the stent so that the second coating 160 has an upper (or outer) layer 161 which covers the upper (or outer) layer 151 of coating 150 and a lower (or inner) layer 162 that completely covers the lower (or inner) layer 152 of coating 150. Both coatings 150 and 160 may be medicinal coatings. Alternately, coating 150 could be primarily an adhesive coating to further increase the adhesion of coating 150 to the stent struts such as strut 114 and which is also particularly adapted to form a tight adhesive bond with second coating 160, which may be a particular medicinal coating that does not bond well if applied directly to the material which comprises the stent strut 114.

It is significant to note that the overall thickness t₃ of the double coated stent of FIG. 6A is significantly less that the thickness to of the uncoated prior art stent of FIG. 1. The use of “flexion” relief cuts allows the use of thinner, wider struts (as shown in Fits. 2 and 3). The reduced thickness t₂ together with the thickness of one or two layers of coating material is still significantly less than t₁ thereby reducing the profile of the stent and maximizing flow through the stented artery (or other lumen).

FIG. 6B shows an alternate embodiment wherein the first coating 150 does not completely fill relief cuts 145 and 146. When second coating 170 is applied, it forms connecting links 175 and 176 which fill the remaining space in relief cuts 145 and 146. Connecting links connect outer layer 171 with inner layer 172 of coating 170 to increase its adhesion to first coating 150.

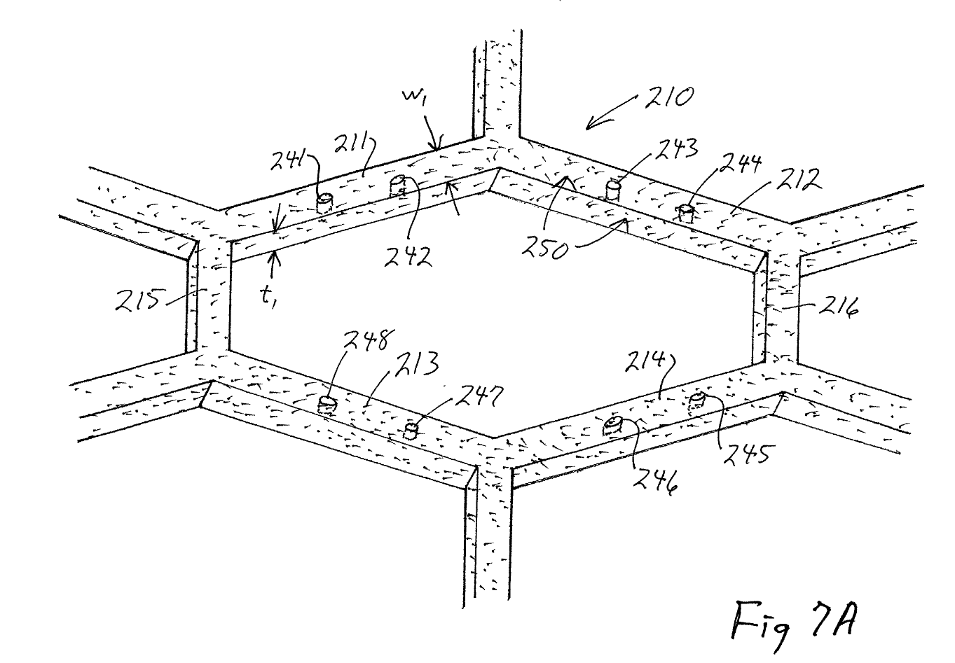

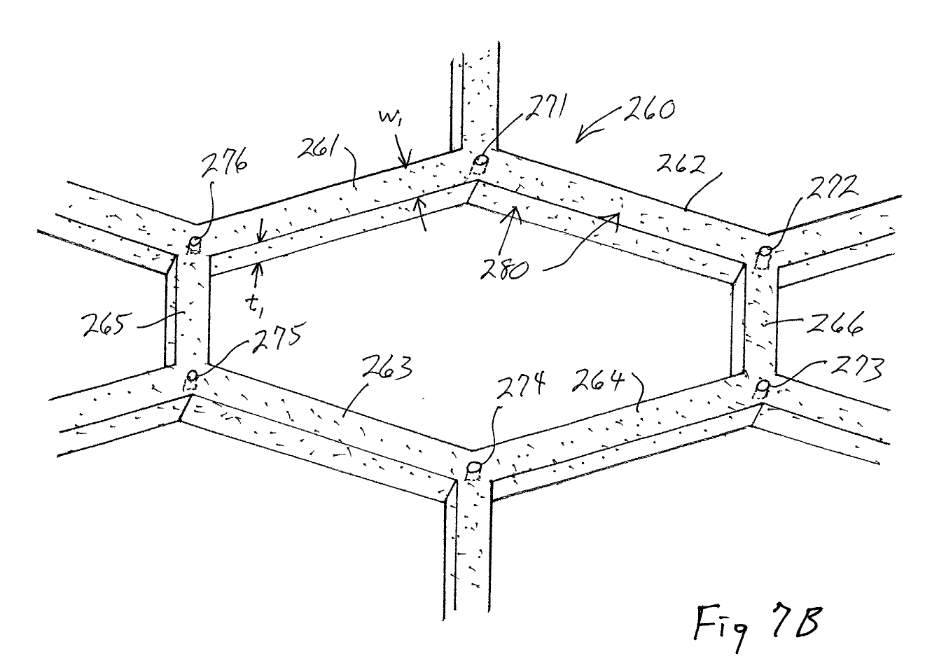

The present invention includes various embodiments. For example, FIG. 7A illustrates a stent cell configuration 210 wherein the struts 211-216 are essentially identical to struts 11-16 of the prior art cell configuration of FIG. 1. Struts 211-216 have the same width w₁ and thickness t₁ as the prior art stent design of FIG. 1. However, FIG. 7A illustrates the use of “lattice support” relief cuts 241-248 which are positioned away from the flexion points and are positioned in the center of struts 211-214. The purpose of the plurality of “lattice support” relief cuts 241-248 is to enhance the adhesion of coating 250 applied to the entire surface of the stent and which also completely fills up each of the relief cuts 241-248. Although the embodiment illustrated in FIG. 7A uses “lattice support” relief cuts to enhance the adhesion of the coating 250, this embodiment is not a preferred form of the invention since it does not include any of the “flexion” relief cuts and therefore utilizes the relatively thick struts 211-216 having thickness t₁.

FIG. 7B shows a cell 260 essentially the same as the prior art cell configuration of FIG. 1 wherein struts 261-266 are the same as struts 11-16. However, FIG. 7B illustrates the use of flexion relief cuts 271-276 without the use of any other relief cuts. Coating 280 covers the entire stent. This is not a preferred form of the invention because it uses the relatively thick struts with thickness t₁.

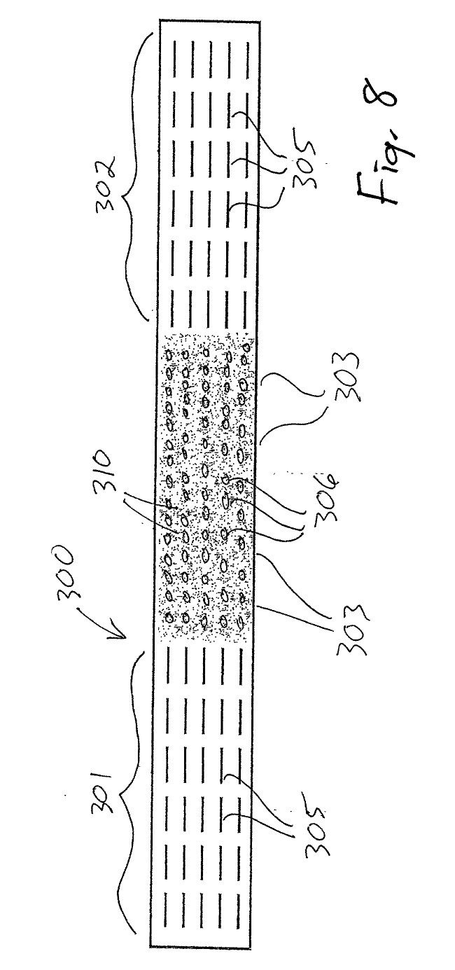

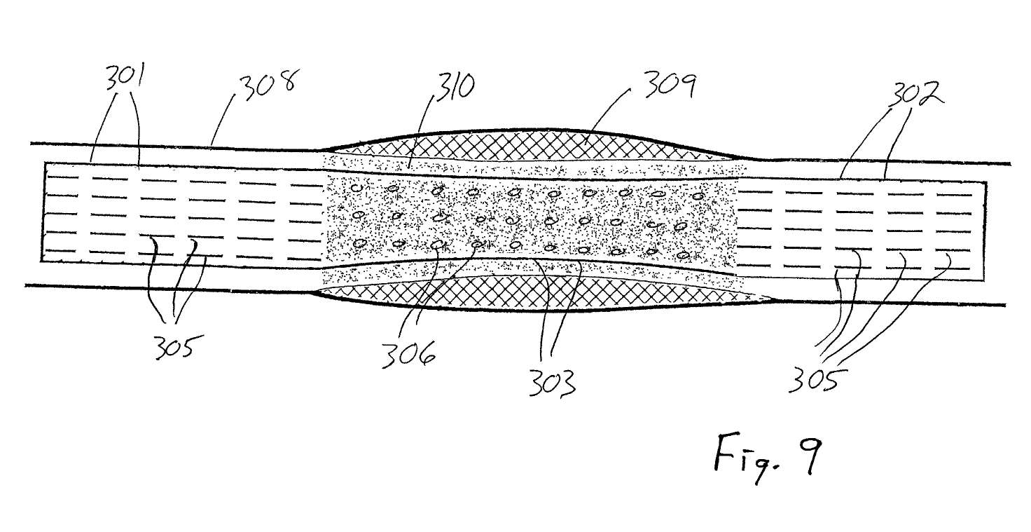

FIGS. 8 and 9 include a further embodiment of the invention as applied to a “dogbone” stent shown generally as 300. Stent 300 has a proximal end 301 and a distal end 302 which are intended to be positioned on opposite sides of a plaque deposit 309 as illustrated in artery 308 shown in FIG. 9. The proximal and distal ends 301 and 302 have an array of “flexion” relief cuts formed therein which are shown schematically by the horizontal lines 305. The central region of the stent 303 has a series of “lattice support” relief cuts formed in the stent. In the embodiment shown in FIGS. 8 and 9, only the central region 303 of the stent has a medicinal coating 310 applied. The proximal and distal ends 301 and 302 do not have medicinal coatings applied thereto. This embodiment is useful, for example, in instances where the high cost of the medicine makes it desirable to limit the region of the stent to which the medicinal coating is applied. The “lattice support” relief cuts are shown schematically as “O's” 306. The proximal and distal ends of the stent expand first and contact the arterial wall 308 on both sides of plaque deposit 309 before the central region of the stent expands. The central region of the stent 303 thereafter expands and contacts the plaque deposit. This sequential expansion reduces the likelihood of pieces of plaque being dislodged from plaque deposit 309 and causing potential serious injury to the patient.

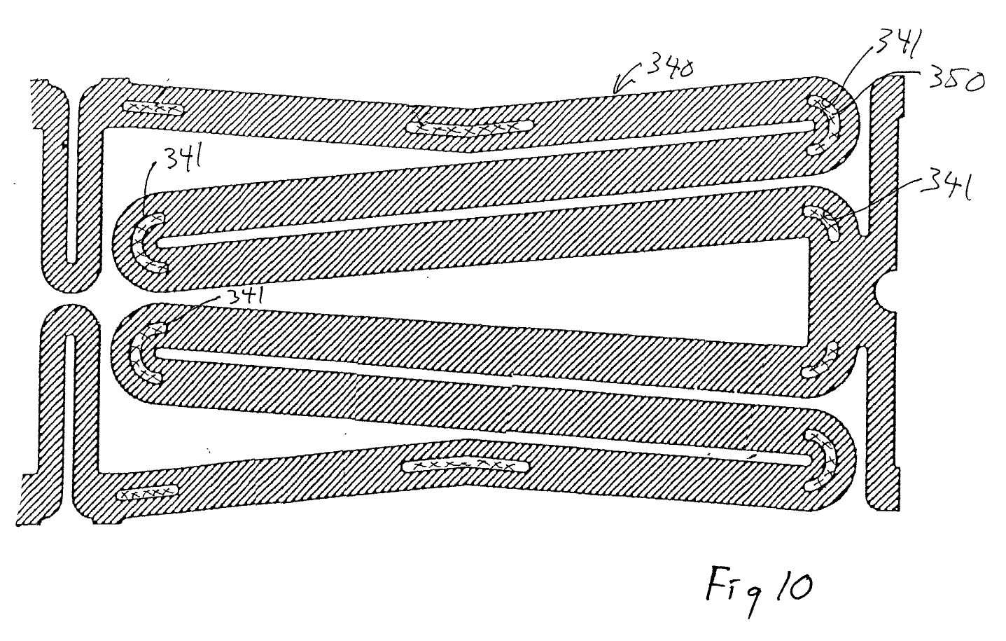

FIG. 10 illustrates yet another embodiment of the invention wherein a stent cell configuration 340 is provided and which is disclosed in greater detail in application Ser. No. 09/357,699 filed Jul. 20, 1999, which is incorporated herein by reference. Relief cuts 341 are formed at flexion points and filled with “plugs” of medicine 350. It is also within the scope of this invention to completely cover the stent 340 with medicinal coating. However, the “plugs” 350 carried within the relief cuts 341 contact the vessel wall as the stent is expanded and remain in contact with the vessel wall after the stent is expanded.

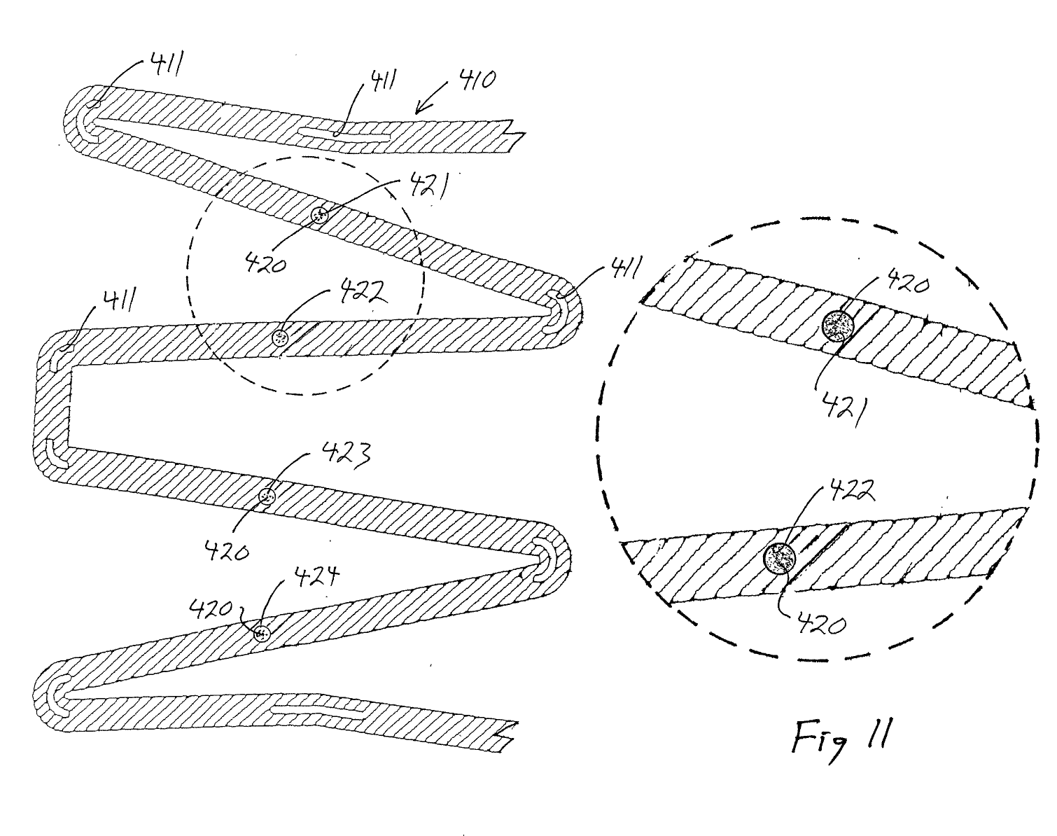

FIG. 11 illustrates a further embodiment of the invention. In this embodiment, stent cell configuration 410 has a plurality of “flexion” relief cuts 411 formed at various flexion points of the stent. In addition, an array of “lattice support” relief cuts 421-424 are also placed on the struts between the flexion points. Stent cell 410 also is shown in FIG. 11 as having a coating 420 applied thereto which fills only the relief cuts 421-424 with “plugs” 420.

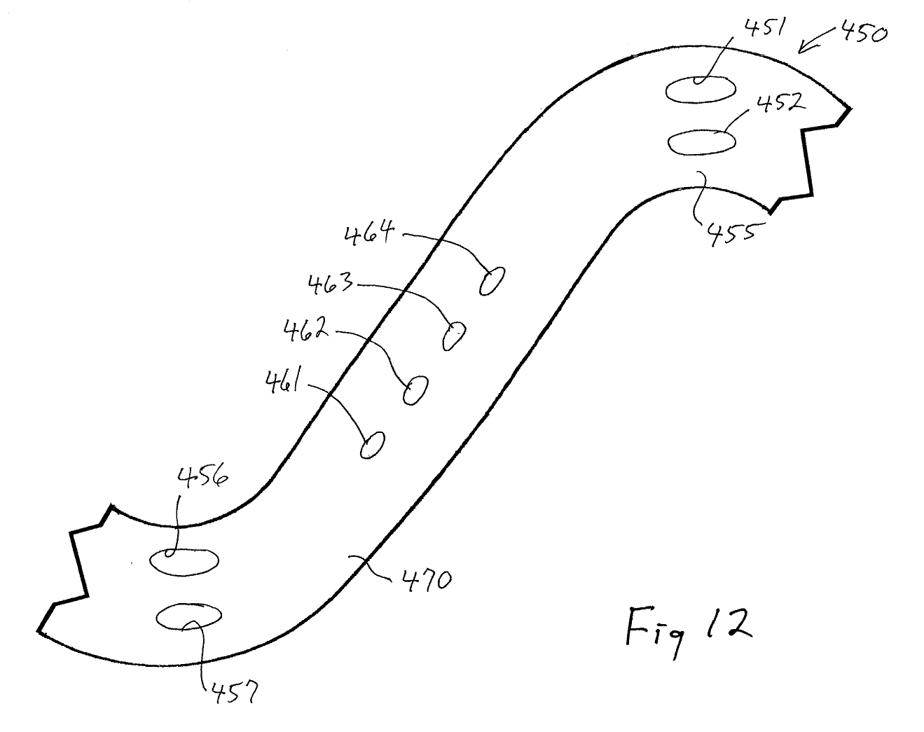

FIG. 12 illustrates yet another embodiment of the invention wherein a stent cell configuration is shown generally as 450. In this embodiment, a pair of elliptical “flexion” relief cuts 451 and 452 are formed at flex point 455 and similarly a pair of elliptical “flexion” relief cuts 456 and 457 are formed at “flexion” point 460. In addition, a series of four smaller elliptical “lattice support” relief cuts 461-464 are formed in strut 470 and are located between flexion points 455 and 460 so as to not significantly alter the manner in which the stent cell 450 expands. The embodiment illustrated in FIG. 12 shows that more than one “flexion” relief cut may be formed in each flex point and that the “flexion” relief cuts may be of a shape other than a circular cylinder. Furthermore, the “lattice support” relief cuts 461-464 may be smaller in shape than the “flexion” relief cuts. The coating is not shown for clarity.

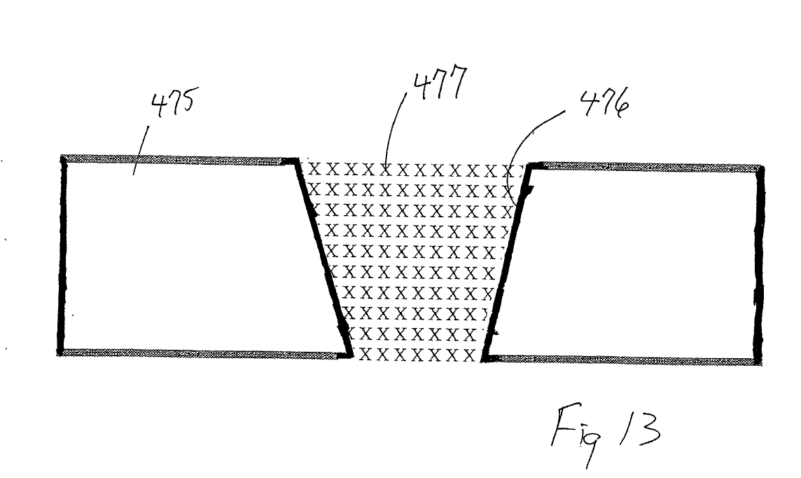

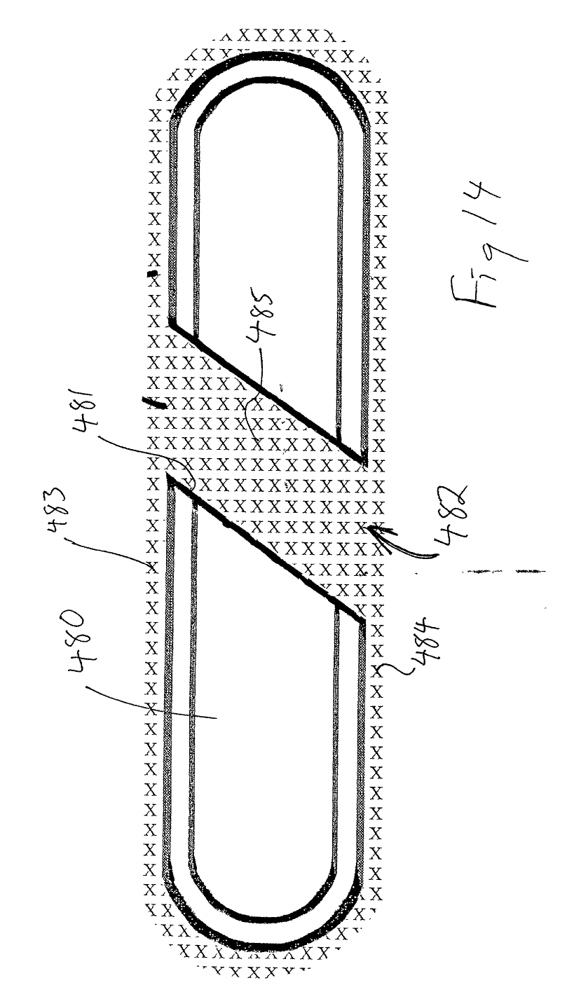

FIGS. 13 and 14 are sectional views along the length of the strut showing alternate shaped relief cuts. FIG. 13 illustrates strut 475 having a tapered, frusto-conical shaped relief cut 476 formed therein. As shown in FIG. 13, a “plug” of medicinal coating 477 is illustrated.

FIG. 14 illustrates strut 480 having an inclined relief cut 481 which has a cylindrical cross section. Medicinal coating 482 has an upper or outer surface 483 and a lower or inner surface 484 which completely cover the outer surface of strut 480. A “plug” of coating material 485 fills up relief cut 481 and integrally connects the outer surface 483 of the coating with the inner surface 484.

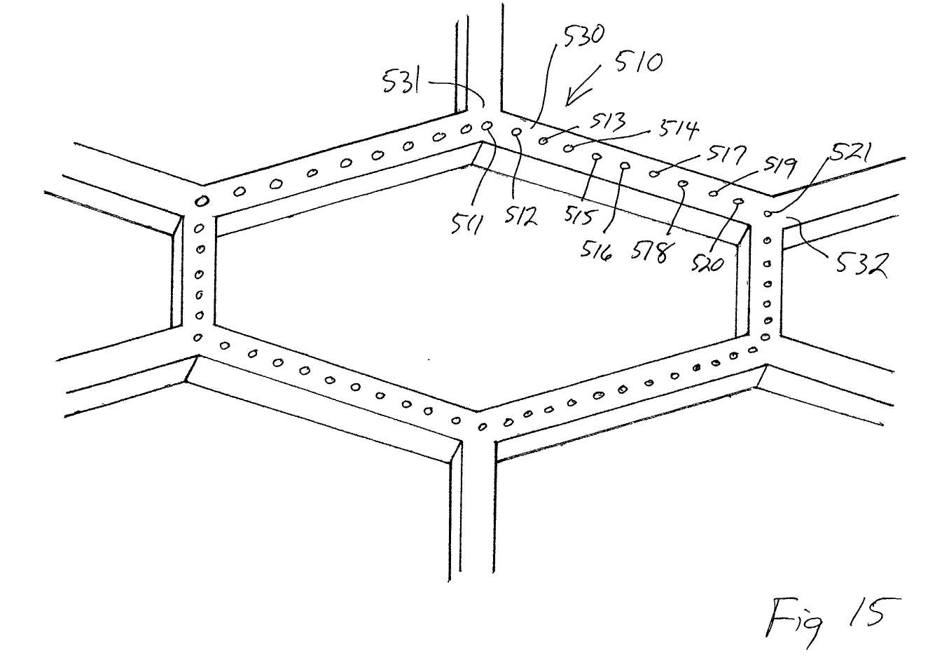

FIGS. 15-17 illustrate additional patterns of relief cuts which are within the scope of the invention. FIG. 15 illustrates a stent cell configuration 510, identical to the prior art stent shown in FIG. 1, but having a relatively large array of smaller relief cuts 511-521 formed in strut 530 which extends between flexion point 531 and 532. Similar relief cuts are formed in the other struts as well. It is significant to note that in this embodiment of the invention some of the relief cuts, such as 512 and 520, may tend to increase the flexibility of the stent even though they are placed at a point away from the primary flexion point of the stent. The primary flexion points of strut 530 are at 531 and 532 and relief cuts 512 and 520 are somewhat removed from those flexion points. However, the array illustrated in FIG. 15 is nevertheless within the scope of the invention. The coating is not shown for clarity.

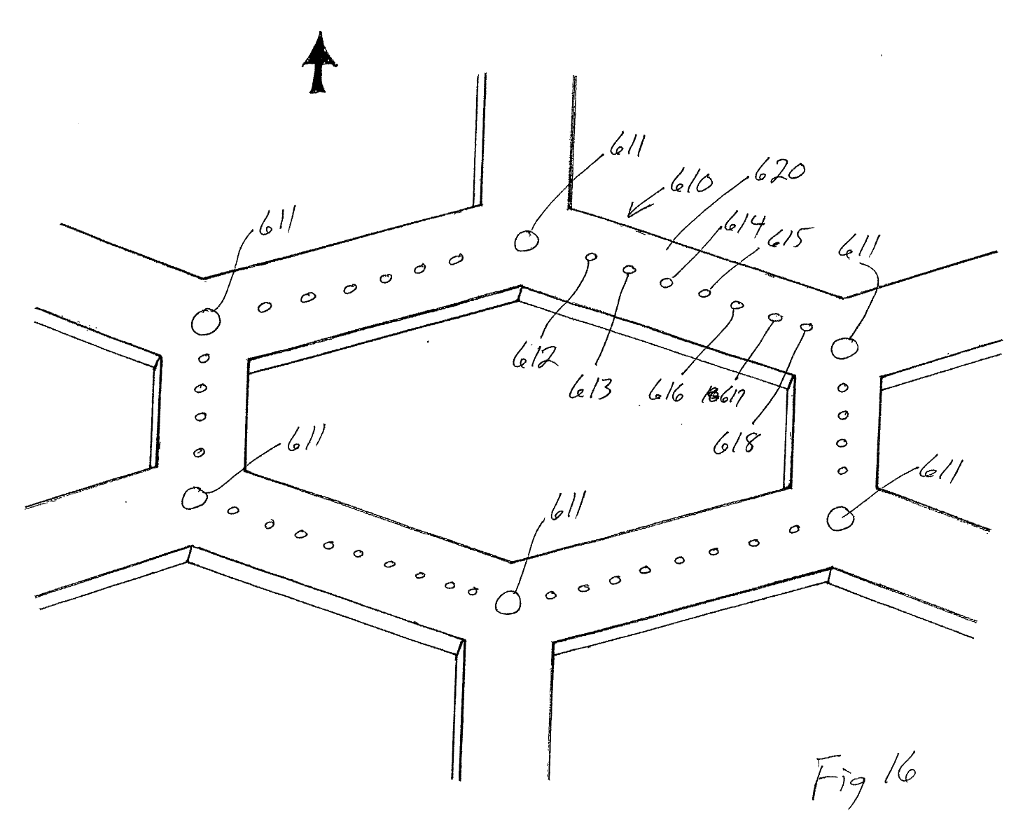

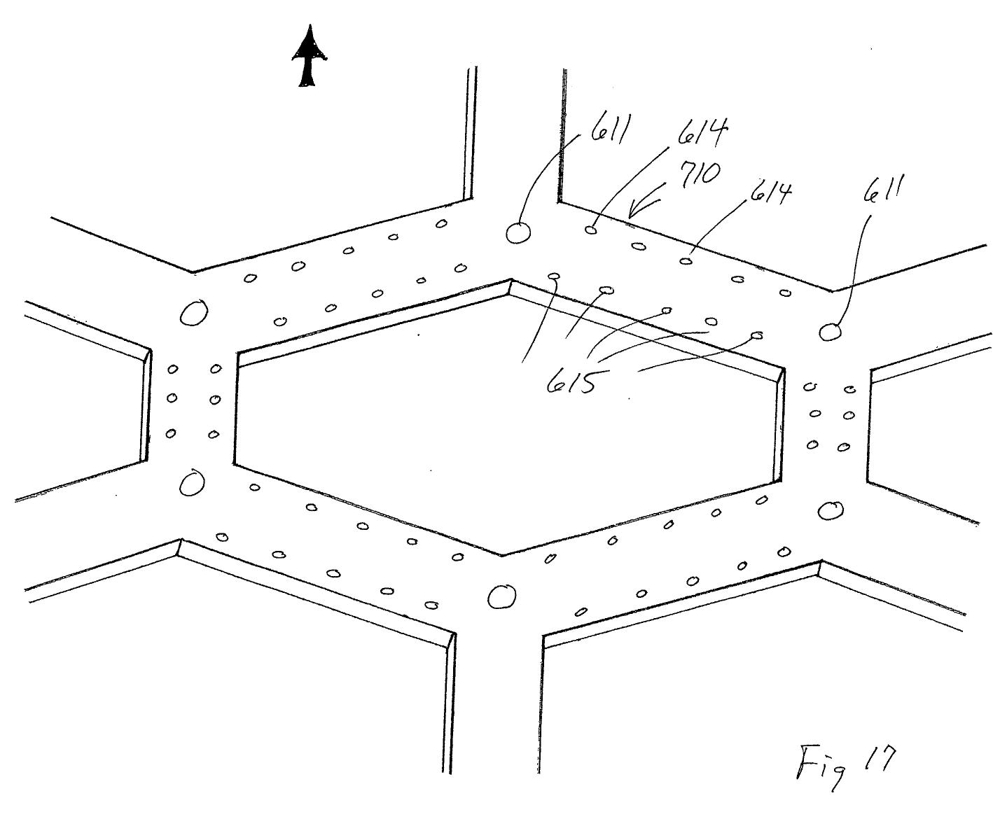

FIGS. 16 and 17 illustrate variations of the relief cut patterns to the stent cell configuration, shown generally in FIG. 2 without a coating applied. FIG. 16 shows a stent cell 610 which includes an array of “flexion” relief cuts 611 formed at each of the flexion points and an array of smaller “lattice support” relief cuts as, for example, 612-618 formed in one of the struts 620. The relief cuts 612 and 618, positioned closest to the flexion points, may contribute somewhat to increasing the flexion of the stent cell 610 and simultaneously provide “lattice support” for a coating to be applied to the surface of the stent. It is within the scope of the invention to include relief cuts that contribute simultaneously to the flexion of the stent and simultaneously contribute to increasing the adhesion of the coating applied to the stent.

FIG. 17 shows yet another stent cell configuration having flexion relief cut 611 and having double rows of “lattice support” relief cuts 614 and 615. This embodiment illustrates that there may be some applications in which placement of a larger number of smaller lattice support relief cuts may advantageously be placed closer to the edges of the stent struts.

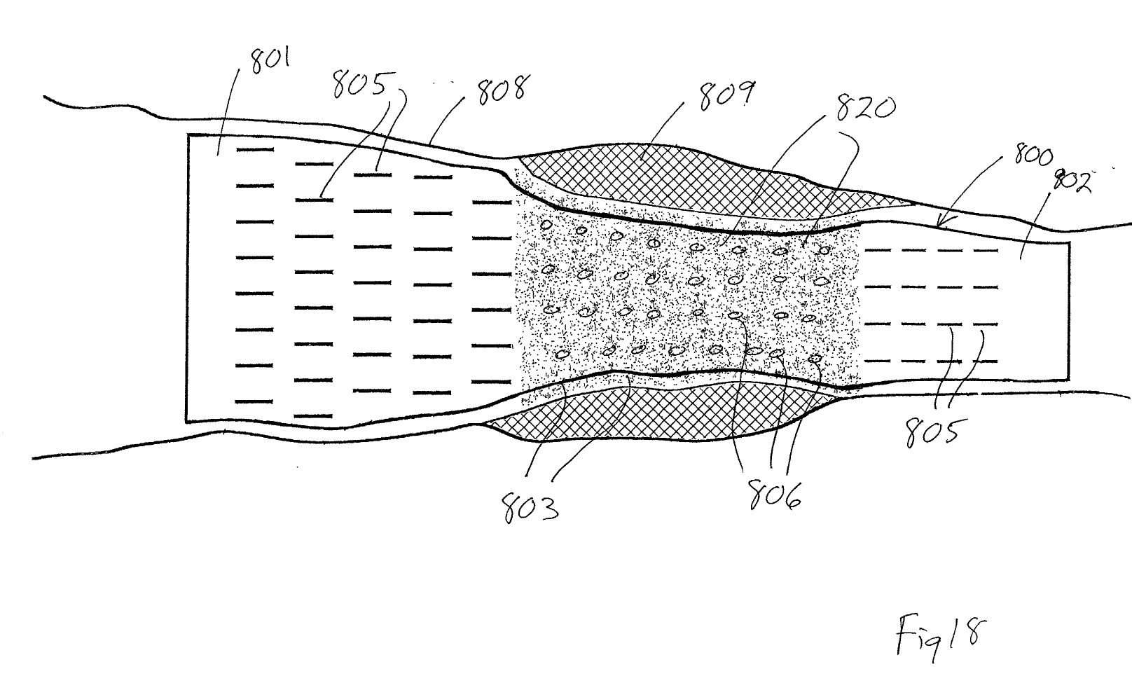

FIG. 18 illustrates a further embodiment of the invention as applied to a tapered artery. Stent 800 has a proximal end 801 and a distal end 802 which are intended to be positioned on opposite sides of a plaque deposit 809 as illustrated in artery 808. The proximal and distal ends 801 and 802 have an array of “flexion” relief cuts formed therein, which are shown schematically by the horizontal lines 805. The central region of the stent 803 has a series of “lattice support” relief cuts shown schematically as 806. Only the central region 803 of the stent has a medicinal coating 820 applied thereto. The proximal and distal ends 801 and 802 expand first and contact the arterial wall 808 on both sides of plaque deposit 809 before the central region 803 of the stent expands.

The relief cuts described herein allow the use of wider and thinner struts without causing the stent to fail prematurely due to fatigue. Placement of the relief cuts as described above allows the stents as described herein to flex and bend during the contraction and expansion of coronary arteries, for example, or other arteries. The relief cuts, according to the present invention, allow the stent to flex and bend after it has been placed in the artery or other lumen without failing from fatigue.

The invention as described herein may also be used in a wide variety of materials. For example, the relief cuts together with coatings may be used on stainless steel, nitinol, plastic and even in conjunction with composite materials.

The foregoing description of the invention has been presented for purposes of illustration and description and is not intended to be exhaustive or to limit the invention to the precise form disclosed. Many modifications and variations are possible in light of the above teaching. The embodiments were chosen and described to best explain the principles of the invention and its practical application to thereby enable others skilled in the art to best use the invention in various embodiments and with various modifications suited to the particular use contemplated. The scope of the invention is defined by the following claims.

What is claimed is:

1. In an expandable stent having inner and outer surfaces, wherein said stent has a plurality of interconnected struts, and wherein said interconnected struts flex relative to each other at flexion points as said stent expands, the improvement comprising:
an array of relief cut means formed in some of said interconnected struts wherein each of said relief cut means extends through said strut and is sufficiently small that the strength of each of said interconnected struts between flexion points is not significantly reduced by the presence of said relief cut means formed therein, and
a coating applied to said stent wherein said coating extends into at least some of said relief cut means.

2. The apparatus of claim 1 wherein said coating covers a portion of said outer surface of said stent.

3. The apparatus of claim 1 wherein said coating is a medicinal coating.

4. The apparatus of claim 1 wherein said interconnected struts of said stent have cross sections wherein the width is greater than the thickness.

5. The apparatus of claim 4 wherein said width is between 1.5 and 5 times as great as said thickness.

6. The apparatus of claim 1 wherein said array of relief cuts includes one or more flexion relief cuts formed at said flexion points and one or more lattice support relief cuts formed between said flexion points.

7. The apparatus of claim 6 wherein said coating covers a portion of said outer and inner surfaces of said stent and extends into one or more of said lattice support relief cuts.

8. The apparatus of claim 1 wherein said array of relief cuts includes only flexion relief cuts formed at said flexion points.

9. The apparatus of claim 1 wherein said array of relief cuts includes only lattice support relief cuts formed between flexion points.

10. The apparatus of claim 9 wherein said coating covers the entire outer surface and inner surface of said stent and extends through said lattice support relief cuts.

11. The apparatus of claim 1 wherein said coating comprises an array of plugs formed in said relief cut means and does not extend onto the inner or outer surface of said stent.

12. The apparatus of claim 1 wherein said coating comprises first and second separate layers of material applied to said stent, said first layer applied to said inner and outer surfaces of said stent, and said second layer being formed on top of said first layer.

13. The apparatus of claim 1 wherein said coating comprises a first layer of material applied to a first region of said stent and a second layer of material applied to a second region of said stent.
